(19) United States
(12) Patent Application Publication
(10) Pub. No.: US 2021/0299068 A1
(43) Pub. Date: Sep. 30, 2021

(54) ANTIMICROBIAL COMPOSITIONS COMPRISING CHLORHEXIDINE

(71) Applicant: 3M INNOVATIVE PROPERTIES COMPANY, SAINT PAUL, MN (US)

(72) Inventors: VINOD P. MENON, WOODBURY, MN (US); JAMES P. DIZIO, SAINT PAUL, MN (US)

(73) Assignee: ., SAINT PAUL, MN (US)

(21) Appl. No.: 17/270,228

(22) PCT Filed: Sep. 9, 2019
(86) PCT No.: PCT/US2019/050143

§ 371 Date: Feb. 22, 2021

Related U.S. Application Data
(60) Provisional application No. 62/731,804, filed on Sep. 14, 2018.

(51) Int. Cl. A61K 31/155; A61K 47/10; A61K 47/38; A61K 47/18

(52) U.S. Cl. CPC A61K 31/155; A61K 47/183; A61K 47/38; A61K 47/10

(57) ABSTRACT

Chlorhexidine gluconate is a commonly used disinfectant that is soluble in aqueous solutions. Because it is a common disinfectant, chlorhexidine gluconate is often used in alcohol solvents, but chlorhexidine gluconate is not soluble in C₂-0₅ alcohol solvents. Also, a chlorhexidine gluconate containing solution can be susceptible to spontaneous calcium gluconate crystallization when formulated with high levels of solvent such as alcohol. These calcium gluconate crystals appear like glass shards. It is desirable to inhibit the formation of these crystals without affecting the antimicrobial potency of the solution. The disclosed chlorhexidine antimicrobial composition includes a chelating agent to inhibit formation of this salt or reverse crystal formation.

### BACKGROUND

It is a standard practice to disinfect the skin prior to any invasive procedure such as surgery, catheterization, or needle puncture to reduce the risk of infection. Currently, chlorhexidine compositions are an agent of choice for disinfecting hands, skin, surgical sites, catheter sites, and oral cavities. Chlorhexidine and its salts are well-known antimicrobials with excellent efficacy that are safe to use. Chlorhexidine and its salts also show persistent antimicrobial activity on the skin often for more than 24 hours.

### SUMMARY

Chlorhexidine gluconate is a commonly used disinfectant that is soluble in aqueous solutions. Because it is a common disinfectant, chlorhexidine gluconate is often used in lower C₂-C₅ alcohol solvents, but chlorhexidine gluconate is not soluble in C₂-C₅ alcohol solvents. Also, a chlorhexidine gluconate containing solution can be susceptible to spontaneous calcium gluconate crystallization when formulated with high levels of solvent such as alcohol. These calcium gluconate crystals appear like glass shards. It is desirable to inhibit the formation of these crystals without affecting the antimicrobial potency of the solution. The disclosed chlorhexidine antimicrobial composition includes a chelating agent to inhibit formation of this salt or reverse crystal formation.

In one embodiment, the antimicrobial composition comprises chlorhexidine, a chlorhexidine-soluble solvent, a chlorhexidine-insoluble solvent, wherein the chlorhexidine-insoluble solvent comprises at least 35 wt. % of the antimicrobial composition, and a chelating agent.

In one embodiment, the chlorhexidine is selected from the group consisting of chlorhexidine digluconate, chlorhexidine diacetate, chlorhexidine dihydrochloride, chlorhexidine dimethosulfate, chlorhexidine dilactate, chlorhexidine diglucoheptonate, chlorhexidine diglycollate salts, and combinations thereof. In one embodiment, the chlorhexidine is present in an amount of least 0.05% by weight based on the total weight of the composition.

In one embodiment, the chlorhexidine-soluble solvent comprises water. In one embodiment, the chlorhexidine-soluble solvent is at least 15 wt. % of the antimicrobial composition. In one embodiment, the chlorhexidine-soluble solvent is less than 25 wt. % of the antimicrobial composition.

In one embodiment, the chlorhexidine-insoluble solvent comprises solvent is a C₂-C₅ lower alcohol. In one embodiment, the chlorhexidine-insoluble solvent comprises a hydrophobic polymer soluble or dispersible in the lower alcohol. In one embodiment, the hydrophobic polymer is selected from the group consisting of acrylates and its derivatives, cellulose and its derivatives, n-vinyl lactam copolymers and vinyl copolymers, and combinations of two or more of the foregoing. In one embodiment, the hydrophobic polymer is present in the antimicrobial composition in an amount of at least 2 wt. % based on the total weight of the antimicrobial composition. In one embodiment, the sec chlorhexidine-insoluble solvent is at least 60 wt. % of the antimicrobial composition. In one embodiment, the chlorhexidine-insoluble solvent is less than 80 wt. % of the antimicrobial composition. In one embodiment, the chlorhexidine-soluble solvent comprises less than 25 wt. % and the chlorhexidine-insoluble solvent comprises at least 75 wt. % of the antimicrobial composition.

In one embodiment, the chelating agent is a polyanionic chelating agent. In one embodiment, the chelating agent is a polycarboxylic acid. In one embodiment, the chelating agent is a soluble in the chlorhexidine-soluble solvent. In one embodiment, the chelating agent is a soluble in the chlorhexidine-insoluble solvent. In one embodiment, the chelating agent has a formation constant with calcium of at least 10⁶ at neutral pH. In one embodiment, the chelating agent is present in the antimicrobial solution between 10 ppm and 10,000 ppm. In one embodiment, the chelating agent is present in the antimicrobial solution less than 100 ppm. In one embodiment, the chelating agent is selected from the group consisting of glutamic acid N,N-diacetic acid, methylglycine N,N-diacetic acid, glucoheptonic acid, ethanoldiglycinic acid, diethylenetriaminepentaacetic acid, nitrilotriacetic acid, (N-(2-hydroxyethyl) ethylenediamine-N,N′,N′-triacetic acid trisodium salt, disodium ethylenediaminetetraacetic acid, or combinations thereof. In one embodiment, the chelating agent removes or prevents the crystallization of at least one of aluminum, barium, iron, calcium, cooper, cobalt, cadmium, mercury, magnesium, manganese, nickel, lead, strontium ions.

In one embodiment, the antimicrobial composition further comprising a plasticizer. In one embodiment, the plasticizer is an emollient ester. In one embodiment, the emollient ester is selected from the group consisting of diesters of bibasic acids, triesters of citric acid, diesters of diols, triesters of triols, and combinations thereof.

In one embodiment, the antimicrobial composition is applied to a surface and is dried on the surface.

### DEFINITIONS

“Ambient temperature” as used herein refers to the temperature range between about 21° and 25° C.

“Chlorhexidine-soluble solvent” as used herein is a solvent where chlorhexidine is substantially soluble at 23° C.

“Chlorhexidine-insoluble solvent” as used herein is a solvent where chlorhexidine is substantially insoluble at 23° C.

“Cidatrope” as used herein is a term for a hydrophobic component in the composition that enhances the effectiveness of the antimicrobial composition such that when the composition less the antimicrobial agent and the composition less the cidatrope component are used separately, they do not provide the same level of antimicrobial activity as the composition as a whole. For example, a cidatrope component in the absence of the antimicrobial agent may not provide any appreciable antimicrobial activity. The enhancing effect can be with respect to the level of kill, the speed of kill, and/or the spectrum of microorganisms killed, and may not be seen for all microorganisms. The cidatrope component may be a synergist such that when combined with the remainder of the composition, the composition as a whole displays an activity that is greater than the sum of the activity of the composition less the cidatrope component and the composition less the antimicrobial agent. The cidatrope typically is a liquid at ambient conditions with a melt temperature less than 25° C. When more than one cidatrope is present in the antimicrobial composition, at least one cidatrope has a melt temperature less than 25° C. The hydrophobic polymer, the emollient esters, and the optional fatty component all function as cidatropes in the compositions described herein.

“Copolymer” includes a polymer of any length (including oligomers) of two or more types of polymerizable monomers, and therefore includes terpolymers, tetrapolymers, etc., which can include random copolymers, block copolymers, or sequential copolymers.

“Emollient” as used herein refers to materials which are capable of maintaining or improving the moisture level, compliance, or appearance of the skin when used repeatedly. Emollients often act to increase the moisture content of the stratum corneum. Emollients are generally separated into two broad classes based on their function. The first class of emollients function by forming an occlusive barrier, which reduces water evaporation from the stratum corneum. The first class of emollients is further subdivided into compounds, which are waxes at room temperature and compounds which are liquid or oils. The second class of emollients penetrate into the stratum corneum and physically bind water to prevent evaporation. The second class of emollients includes those that are water soluble and are often referred to as humectants. The emollient esters are considered separate and distinct from any other emollients which may be used, even though the emollient esters may function as occlusive emollients and aid in maintaining or improving the skin condition.

“Essentially free” means less than 1% by weight, in one embodiment less than 0.5% by weight, and in one embodiment less than 0.1% by weight, of a component based on the total weight of the composition.

“Fatty” as used herein refers to a hydrocarbon chain length of 8 or more carbon atoms (odd or even number), unless otherwise specified.

“Hydrophobic” or “water insoluble” refers to a material that will not significantly dissolve in water at 23° C.

“Hydrophobic polymers” as disclosed have a solubility in water of less than 1%, in one embodiment less than 0.5%, in one embodiment less than 0.25%, and in one embodiment less than 0.10%.

“Hydrophilic” or “water soluble” or “water swellable” refers to a material that will dissolve, solubilize, disperse or otherwise suspend in water (or other aqueous solution as specified) at a temperature of 23° C. in an amount of at least 7% by weight, in one embodiment at least 10% by weight, in one embodiment at least 20% by weight, in one embodiment at least 25% by weight, in one embodiment at least 30% by weight, and in one embodiment at least 40% by weight, based on the total weight of the hydrophilic material and the water. The component is considered dissolved if after thoroughly mixing the compound with water at 60° C. for at least 4 hours and allowing this to cool to 23-25° C. for 24 hours, and mixing the composition thoroughly it appears uniform clear solution without visible cloudiness, phase separation, or precipitate in a jar having a path length of 4 cm. Typically, when placed in 1×1 cm cell, the sample exhibits greater than 70% transmission measured in a suitable spectrophotometer at a wavelength of 655 nm. Water dispersible hydrophilic materials disperse in water to form uniform cloudy dispersions after vigorous shaking of a 5% by weight mixture of the hydrophilic component in water. Water swellable hydrophilic materials solubilize or suspend in water, including those materials that form of a viscous solution or viscous gel.

“Lotion” means liquid or cream, free of any propellant.

“(Meth)acrylate monomers” are acrylic acid esters or methacrylic acid esters of alcohols.

“Nonvolatile” means that the component does not evaporate readily at ambient conditions, such that a 20 gm sample in a 4 cm² dish does not lose more than 2% of its weight, e.g., within 60 minutes upon exposure to ambient conditions. Examples of nonvolatile components of the compositions described herein include glycerin, chlorhexidine and its salts, and fatty components with a chain length greater than 10 carbons.

“Polymer” as used herein refers to a natural or synthetic molecule having repetitive units and a number average molecular weight of at least 10,000 and includes homopolymers and copolymers of any length.

“Solubility” can be determined by thoroughly mixing the compound with the solvent at the appropriate concentration at 23° C. for at least 24 hours (or at elevated temperature if that is necessary to dissolve the compound), allowing this to sit at 23-25° C. for 24 hours, and observing the sample. In a glass jar with a 4-cm path length the sample should have evidence of a second phase, which can be liquid or solid and may be separated on the top, bottom, or distributed throughout the sample. For crystalline compounds care should be taken to avoid producing a supersaturated solution. The components should be mixed and observed. Cloudiness or presence of a visible precipitate or separate phase indicates that the solubility limit has been exceeded. Typically, when placed in 1×1 cm cell the sample has less than 70% transmission measured in a suitable spectrophotometer at a wavelength of 655 nm. For solubility determinations less than that which can be observed with the naked eye the solubility is determined using radiolabeled compounds as described under “Conventional Solubility Estimations in Solubility of Long-Chain Fatty Acids in Phosphate Buffer at pH 7.4,” Henrik Vorum, et al. in Biochimica et. Biophysica Acta, 1126, 135-142 (1992).

“Solvent” as used herein refers to any compound used to dissolve or disperse another compound.

“Solvent system” or “hydroalcoholic solvent system” as used herein refer to the combination of the chlorhexidine-soluble solvent and chlorhexidine-insoluble solvent in the compositions described herein.

“Surfactant” as used herein is synonymous with “emulsifier,” and means an amphiphile (a molecule possessing both polar and nonpolar regions which are covalently bound) capable of reducing the surface tension of water and/or the interfacial tension between water and an immiscible liquid.

### DETAILED DESCRIPTION

The compositions provided herein comprise chlorhexidine and a chlorhexidine-soluble solvent and a chlorhexidine-insoluble solvent. Chlorhexidine has limited solubility in alcoholic solutions. In some embodiments, relatively high alcoholic concentrations are desired. In some embodiments, the alcohol solutions further comprise additional components, such as hydrophobic polymers and plasticizers so that following drying, a chlorhexidine-containing film is formed.

Inclusion of polymers and plasticizer further increase the concentration of the chlorhexidine-insoluble solvent. When the concentration of the chlorhexidine-insoluble solvent is at least 35 wt % of the total composition, over time spontaneous crystal formation can occur from the chlorhexidine that appear like glass or small needles. The addition of a small amount of a chelator can completely reverse the crystal formation and resolubilize the crystal through highly effective and specific chelation or prevent formation of the crystal.

### Chlorhexidine

The chlorhexidine is that component of the composition that provides at least part of the antimicrobial activity. The chlorhexidine comprises chlorhexidine digluconate, chlorhexidine diacetate, chlorhexidine dihydrochloride, chlorhexidine dimethosulfate, chlorhexidine dilactate, chlorhexidine diglucoheptonate, chlorhexidine diglycollate salts, and combinations thereof.

Based on the total weight of the antimicrobial composition, the chlorhexidine can be used at levels of at least 0.05% by weight, in one embodiment at least 0.1% by weight and in one embodiment at least 0.25% by weight and in one embodiment at least 0.5% by weight. Compounds of this class are typically used at levels less than about 8% by weight, in one embodiment less than about 6% by weight, and in one embodiment than about 4% by weight of the composition.

The chlorhexidine may be present as the free base or as a disalt of acetate, gluconate, lactate, methosulfate (CH₃OSO₃⁻), or a halide or combinations thereof. A commonly used chlorhexidine is chlorhexidine digluconate (CHG).

Care must also be taken when formulating chlorhexidine to avoid inactivation by sequestering it in micelles which may be formed by incorporation of surfactants and/or emulsifiers. Typically, compositions are essentially free of surfactants and/or emulsifiers.

Chlorhexidine is very basic and capable of forming multiple ionic bonds with anionic materials. For this reason, chlorhexidine-containing compositions are typically free of anionic compounds that can result in precipitation of the antimicrobial. Anionic surfactants useful, for example, as wetting agents, may also need to be avoided. Halide salts may need to be avoided. For example, chlorhexidine digluconate (CHG) will precipitate rapidly in the presence of halide salts above a concentration of about 0.1M. Therefore, if a system includes CHG, and needs to comprise salts for stability or other purposes, gluconate salts such as triethanolamine gluconate or sodium gluconate, are used.

### Chlorhexidine-Soluble Solvent

The chlorhexidine-soluble solvent can be any solvent where chlorhexidine is substantially soluble in the solvent. In one embodiment, the chlorhexidine-soluble solvent is water. As the chlorhexidine-insoluble solvent increases, the chlorhexidine-soluble solvent decreases. In some embodiments the chlorhexidine-soluble solvent is at least 15 wt % of the total composition. In some embodiments the chlorhexidine-soluble solvent is less than 25 wt % of the total composition.

### Chlorhexidine-Insoluble Solvent

The chlorhexidine-insoluble solvent can be any solvent where the chlorhexidine is substantially insoluble in the solvent. In one embodiment, the chlorhexidine-insoluble solvent is a C₂-C₅ alcohol. The alcohol may be chosen from ethanol and isopropanol. Ethanol is a broad spectrum and quick kill of microbes and an odor acceptable to consumers such as doctors, nurses and clinicians. Propyl alcohols (1-propanol and 2-propanol) may also be used.

A blend of two or more lower alcohols may be used as the chlorhexidine-insoluble solvent in the hydroalcoholic solvent system. The lower alcohols may be denatured, such as for example, denatured ethanol including SDA-3C (commercially available from Eastman Chemical, Kingsport, Tenn.). Co-solvents may be further included in the composition with the lower alcohol. Considering the topical application contemplated for the antimicrobial composition, suitable co-solvents include acetone, hydrocarbons such as isooctane, glycols, ketones, ethers, and short chain esters.

If a hydrophobic polymer or plasticizer are included as part of the chlorhexidine-insoluble solvent, the C₂-C₅ lower alcohol used in the compositions is used in sufficient amount to dissolve the hydrophobic polymer and emollient ester.

In one embodiment, the chlorhexidine-insoluble solvent is present in an amount of at least 35 wt-% of the total composition. In one embodiment, the chlorhexidine-insoluble solvent is present in an amount of at least 60 wt-% of the total composition. In one embodiment, the chlorhexidine-insoluble solvent is present in an amount of at least 80 wt-% of the total composition

Compositions having higher alcohol to water ratios within the range 40:60 to 95:5 ensure an efficacious immediate bacterial kill. In one embodiment the higher alcohol:water ratio is between about 55:45 and 90:10, and in one embodiment at least 65:35. Higher alcohol to water ratios are used in an embodiment for optimum antimicrobial activity and to ensure the composition is fast drying.

A useful concentration of the hydrophobic polymer and the antimicrobial agent depend on their respective solubilities in a given hydroalcoholic solvent system. For example, the solubility of CHG in the hydroalcoholic solvent system decreases with increasing C₂-C₅ alcohol concentration. In contrast, the hydrophobic polymers may require increased levels of C₂-C₅ alcohol concentration to solubilize the hydrophobic polymers. One skilled in the art can readily determine an optimum range of concentrations based on the solubility of the cationic antimicrobial agent and the hydrophobic polymer for a given antimicrobial composition or a given solvent system.

### Chelator

The antimicrobial composition comprises a chelating agent. In one embodiment, the chelating agent is a polyanionic chelating agent. In one embodiment, the chelating agent is a polycarboxylic acid. In one embodiment, the chelating agent is a soluble in the chlorhexidine-soluble solvent. In one embodiment, the chelating agent is a soluble in the chlorhexidine-insoluble solvent. In one embodiment, the chelating agent has a formation constant with calcium of at least 10⁶ at neutral pH. In one embodiment the chelating agent is present in the antimicrobial solution between 10 and 10,000 ppm of the total composition. In one embodiment the chelating agent is present in the antimicrobial solution by less than 100 ppm.

The chelating agent is selected from the group consisting of glutamic acid N,N-diacetic acid, methylglycine N,N-diacetic acid, glucoheptonic acid, ethanoldiglycinic acid, diethylenetriaminepentaacetic acid, nitrilotriacetic acid, (N-(2-hydroxyethyl) ethylenediamine-N,N′,N′-triacetic acid trisodium salt (Trisodium HEDTA), disodium ethylenediaminetetraacetic acid or combinations thereof. The chelating agent removes or prevents the crystallization of at least one of aluminum, barium, iron, calcium, cooper, cobalt, cadmium, mercury, magnesium, manganese, nickel, lead, strontium ions.

Antimicrobial compositions comprising chlorhexidine, chlorhexidine-soluble solvent, chlorhexidine-insoluble solvent that is at least 35 wt % of the antimicrobial composition with a chelator showed effective reduction of crystals.

In one embodiment, the formation constant for the HEDTA complex with calcium is 10⁶ at neutral pH, this high affinity causes a remarkable reversal in calcium gluconate crystal formation at extremely low use levels as can be observed in the attached visuals.

### Optional Hydrophobic Polymers

The antimicrobial composition may include a hydrophobic polymer soluble in the chlorhexidine-insoluble solvent and with a plasticizer, such as an emollient ester, to provides improved antimicrobial efficacy to the antimicrobial composition. For certain embodiments, the hydrophobic polymers have a solubility in water of less than 1%, in one embodiment less than 0.5%, in one embodiment less than 0.25%, and in one embodiment less than 0.10%. Films formed after drying the antimicrobial composition adhere well to the skin, remain flexible and do not crack when the skin is gently flexed, and do not wash off when exposed to water or body fluids.

Hydrophobic polymers suitable for use in the antimicrobial compositions include film-forming polymers derived from n-vinyl lactam, such as those described in U.S. Pat. Nos. 4,542,012 and 4,584,192; vinyl polymers as described in U.S. Pat. No. 7, 030,203; and cellulose, including its derivatives (other than those that are hydrophilic, water soluble or swellable in water), such as ethyl cellulose.

Suitable hydrophobic polymers include film-forming polymers that are the reaction product of a prepolymer having a plurality of isocyanate functionalities, and a polyvinylpyrrolidone polymer. The polyvinylpyrrolidone polymer is a free-radical-polymerization reaction product of at least N-vinylpyrrolidone and a vinyl-functional compound, as further described in U.S. Pat. No. 4,542,012. Other suitable film-forming polymers include film-forming copolymers comprising (i) a monomeric acrylic or methacrylic acid ester of an alkyl alcohol having from 2 to about 14 carbon atoms and containing a single hydroxyl, (ii) a monomeric methacrylic acid ester of an alkyl alcohol having from 1 to 6 carbon atoms and containing a single hydroxyl, and (iii) an N-vinyl lactam, as further described in U.S. Pat. No. 4,584,192.

Other suitable hydrophobic polymers include vinyl polymers, for example, polymers derived from vinyl monomers such as (meth)acrylates, (meth)acrylamides, vinyl ethers, vinyl acetates and their hydrolyzed derivatives, styrenic compounds (i.e., derivatives of styrene), and N-vinyl lactams (including, for example, N-vinylpyrrolidone, N-vinylcaprolactam, and their derivatives). Suitable vinyl polymers are soluble (i.e., form transparent homogenous solutions) or dispersible in the lower alcohol and tend to be insoluble or sparingly soluble in water. Certain vinyl polymers using combinations of three monomers (terpolymers) are also useful.

A class of polymers useful in the antimicrobial compositions described herein include polymers derived from the polymerization of at least one monoethylenically unsaturated alkyl (meth)acrylic monomer, preferably, an alkyl (meth)acrylic acid ester (i.e., an alkyl acrylate or alkyl methacrylate). One class of vinyl polymers contains at least one copolymerized monoethylenically unsaturated alkyl (meth)acrylic monomer. As used herein, the “monoethylenically unsaturated” term in the alkyl (meth)acrylic monomer refers to the acrylic unsaturation. “Alkyl (meth)acrylic” monomers include (meth)acrylamides (e.g., octylacrylamide), (meth)acrylates, and combinations thereof The alkyl (meth)acrylic monomer is an alkyl (meth)acrylic acid ester (i.e., an alkyl acrylate or alkyl methacrylate), wherein the alkyl group has at least 4 carbon atoms (on average).

Examples of monomers which may be used to make the hydrophobic polymer include but are not limited to: vinyl pyridine, methyl acrylate, ethyl acrylate, butyl acrylate, ethylhexyl acrylate, isooctyl acrylate, isoamyl acrylate, isobornyl acrylate, isotetradecyl acrylate, lauryl acrylate, stearyl acrylate, behenyl acrylate, ethyl hexyl diglycol acrylate, 2-hydroxy-3-phenoxypropyl acrylate, hydroxybutyl acrylate, hydroxyethyl acrylate, hydroxypropyl acrylate, butoxyethyl acrylate, ethoxy diethyleneglycol acrylate, hexyl polyethyleneglycol acrylate, methoxy triethyleneglycol acrylate, phenoxyethyl acrylate, phenoxy polyethyleneglycol acrylate, tetrahydrofurfuryl acrylate, glycidyl methacrylate, trimethylpropane benzoate acrylate, methyl methacrylate, ethyl methacrylate, butyl methacrylate, octadecyl acrylate, hydroxypropyl methacrylate, hydroxyethyl methacrylate, vinyl acetate, N-vinylpyrrolidone, N-vinyllactams, styrene, styrene macromer, vinyl butyral, acrylamide, dimethylaminoethyl methacrylate, dimethylamino ethylacrylate, diethylamino ethylstyrene, diethylaminoethyl methacrylate, butylaminoethyl methacrylate, aminoethyl methacrylate hydrochloride, diisopropylaminoethyl methacrylate, morpholinoethyl acrylate, morpholinoethyl methacrylate, dimethylaminoneopentyl acrylate, diallylamine, aminoethyl methacrylamide, aminopropyl methacrylamide, dimethylaminopropyl acrylamide, dimethylaminopropyl methacrylamide, dimethylaminoethyl acrylate, dimethylaminoethyl methacrylamide, and their quaternary salts such as dimethylaminoethyl acrylate methylchloride, diallyldimethylammonium chloride, aminopropyl methacrylamide hydrochloride, aminoethyl methacrylamide hydrochloride. The hydrophobic polymer derived from the polymerization of at least one of these monomers may be a homopolymer, copolymer, terpolymer, or a blend of polymers.

Other suitable hydrophobic polymers include cellulose and its hydrophobic derivatives, for example, methyl, ethyl, propyl, and butyl, optionally including hydroxyl, methoxy, ethoxy, propoxy, and butoxy groups, as well as C₅-C₂₀ alkyl derivatives and derivatives which are a combination thereof. Some examples of such cellulose derivatives include methylhydroxypropylcellulose, cetylhydroxyethylcellulose, hydroxypropylcellulose, ethylhydroxyethylcellulose, ethylcellulose, hydroxymethylcellulose and hydroxybutylmethylcellulose. In one embodiment, the cellulose derivative is ethyl cellulose.

Hydrophobic polymers useful in the antimicrobial compositions described herein are soluble in the hydroalcoholic solvent system, and particularly the chlorhexidine-insoluble solvent, such as a lower alcohol. In general, the hydrophobic polymers used herein are insoluble or only sparingly soluble in water. When used alone, the hydrophobic polymers can be capable of forming water-resistant films. Such polymers are desirable in the antimicrobial compositions described herein because they would produce surgical hand preparations and antimicrobial hand lotions, for example, that cannot be easily washed off with water after being applied and dried.

The hydrophobic polymer of the composition, along with the plasticizer, such as an emollient ester, and optionally the fatty component, can also contribute to the improved adhesion of medical adhesive articles to the skin, particularly in the presence of moisture or fluids. The hydrophobic polymer may be liquid to improve the overall cosmetic skin feel of the composition as well.

The hydrophobic polymers typically are not ethoxylated. Ethoxylation affects the moisture sensitivity of the resultant antimicrobial composition, with a resulting decrease in adhesion performance. If any one of the components is ethoxylated, it is typically no more than one or two moles of ethylene oxide.

When used, the hydrophobic polymer is present in the composition in an amount of at least 0.1 wt-%, in one embodiment at least 1 wt-%, in one embodiment at least 3 wt-%, and in one embodiment at least 5 wt-% based on the total weight of the antimicrobial composition. In certain embodiments, the hydrophobic polymer is present in amounts of no more than 10 wt-%, and in one embodiment no more than 6 wt-%.

### Optional Plasticizer

When the antimicrobial composition includes a hydrophobic polymer, a plasticizer is typically included to provide improved antimicrobial efficacy to the antimicrobial composition. In one embodiment, the plasticizer is an emollient ester, such as a cidatrope that provides improved antimicrobial efficacy to the antimicrobial composition. In one embodiment, the emollient ester comprises a total of at least 8 carbon atoms. In one embodiment, the emollient ester comprises no more than 20 carbon atoms. In one embodiment, the emollient ester comprises at least two ester linkages.

The emollient esters may serve to prevent skin irritation and drying, improve the cosmetic feel of the formulation, enhance the antimicrobial activity of the formulation, and moisturize the skin by reducing water transmission. When used at higher concentrations, the emollient esters also enhance the dry adhesion of medical adhesive articles.

The emollient ester is generally a liquid at room temperature and has poor solubility in water, i.e., soluble in water at 23° C. in amounts less than 2 wt-%. Emollient esters suitable for use as a cidatrope in the antimicrobial compositions are selected from diesters of bibasic acids, diesters of diols, triesters of citric acid, triesters of triols, and combinations thereof.

For certain embodiments, the emollient ester is selected from the group consisting of (C1-C8)alkyl alcohol esters of (C2-C12)diacids, for example, dibutyl adipate, diisopropyl adipate, diisobutyl adipate, dihexyl adipate, diisopropyl sebacate, and dibutyl sebacate; diesters of butanediol and hexanediol; propylene glycol dicaprylate; (C2-C8)alkyl alcohol di and triesters of citric acid, for example, tributyl citrate; and combinations thereof. Other emollient esters include dialkyl acid esters of diols, triesters of citric acid, and trialkyl acid esters of triols, and dialklyl alcohol esters of other di and tri carboxylic acids.

For certain embodiments, the emollient ester is selected from the group consisting of dialkyl esters of bibasic acids, trialkyl esters of citric acid, dialkyl esters of diols, trialkyl esters of triols, and combinations thereof. Diesters of bibasic acids include dibutyl adipate, diisopropyl adipate, diisobutyl adipate, dihexyl adipate, diisopropyl sebacate, dibutyl sebacate and mixtures thereof. In a similar manner, triesters of citric acid include tributyl citrate. Diesters of diols include esters of butanediol and hexanediol. Diesters of propylene glycol such as propylene glycol dicaprylate may also be useful. Typical emollient esters are diisopropyl adipate, dibutyl adipate, and tributyl citrate.

Examples of other emollients that may be suitable include, but are not limited to, short chain (i.e, C1-C6) alkyl or (C6-C12)aryl esters of long (i.e., C8-C36) straight or branched chain alkyl or alkenyl alcohols or acids; short chain (i.e., C1-C6) alkyl or (C6-C12)aryl esters of (C4-C12)diacids or (C4-C12)diols optionally substituted in available positions by —OH; (C2-C18)alkyl or (C6-C12)aryl esters of glycerol, pentaerythritol, ethylene glycol, propylene glycol; (C12-C22)alkyl esters or (C12-C22)ethers of polypropylene glycol; (C12-C22)alkyl esters or (C12-C22)ethers of polypropylene glycol/polyethylene glycol copolymer; and long chain (i.e., C8-C36) alkyl and alkenyl esters of long (i.e., C8-C18) straight or branched chain alkyl or alkenyl alcohols or acids, long chain (i.e., C8-C36) alkyl and alkenyl amides of long straight or branched chain (i.e., C8-C36) alkyl or alkenyl amines or acids.

For certain embodiments, the emollient ester is selected from the group consisting of (C1-C6)alkyl and (C6-C12)aryl esters of (C8-C36) straight or branched chain alkyl or alkenyl alcohols or acids; (C1-C6)alkyl and (C6-C12)aryl diesters of (C2-C12)diacids or (C4-C12)diols, optionally substituted in at least one available position by —OH; (C1-C6)alkyl and (C6-C12)aryl di- or tri-esters of citric acid, (C2-C18)alkyl and (C6-C12)aryl esters of glycerol, pentaerythritol, ethylene glycol, or propylene glycol; (C12-C22)alkyl esters and (C12-C22)ethers of polypropylene glycol; (C12-C22)alkyl esters and (C12-C22)ethers of polypropylene glycol/polyethylene glycol copolymer; long chain (i.e., C8-C36) alkyl and alkenyl esters of long (i.e., C8-C18) straight or branched chain alkyl or alkenyl alcohols or acids, and long chain (i.e., C8-C36) alkyl and alkenyl amides of long straight or branched chain (i.e., C8-C36) alkyl or alkenyl amines or acids.

For certain embodiments, the emollient ester is selected from the group consisting of (C1-C6)alkyl and (C6-C12)aryl esters of (C8-C36) straight or branched chain alkyl or alkenyl alcohols or acids; (C1-C6)alkyl and (C6-C12)aryl diesters of (C2-C12) diacids or (C4-C12)diols, optionally substituted in at least one available position by —OH; and (C1-C6)alkyl and (C6-C12)aryl di- or tri-esters of citric acid.

In one embodiment, the emollient ester is present in the composition in an amount of at least 0.1 wt-%, in one embodiment at least 1 wt-%, and in one embodiment at least 2 wt-%. In embodiments, the emollient ester is present in amounts of no more than 10.0 wt-%, in one embodiment no more than 6 wt-%. Higher levels can be used depending on the ratio of cationic antimicrobial agent to total nonvolatile components as discussed above.

### Optional Fatty Component

The antimicrobial composition can also optionally include a fatty component that provides improved antimicrobial efficacy to the antimicrobial composition. Fatty components include a C₁₂-C₂₁ fatty alcohol, a C₁₂-C₂₁ fatty ester containing one or more free hydroxyl groups, a C₁₂-C₂₁ fatty ether containing one or more free hydroxyl groups, a C₁₂-C₂₁ fatty amide containing one or more free hydroxyl groups, and combinations thereof The fatty component of the composition, along with the hydrophobic polymer and emollient ester, can also contribute to the improved adhesion of medical adhesive articles to the skin, particularly in the presence of moisture or fluids. The fatty component may be waxy to improve the overall cosmetic skin feel of the composition as well.

The fatty components are typically not ethoxylated. Ethoxylation affects the moisture sensitivity of the resultant antimicrobial composition, with a resulting decrease in adhesion performance. If any one of the components is ethoxylated, it is typically no more than one or two moles of ethylene oxide.

When used, the fatty component is present in the composition in an amount of at least 0.5 wt-%, in one embodiment at least 1 wt-%, in one embodiment at least 2 wt-%, and in one embodiment at least 3 wt-% based on the total weight of the antimicrobial composition. In certain embodiments, the fatty component is present in amounts of no more than 6 wt-%, and in some embodiments no more than 5 wt-%.

### Additional Optional Ingredients

The compositions may optionally include ingredients such as salts, humectants (in minimal amounts due to their hydrophilic nature and affect on moisture sensitivity), stabilizers, other antimicrobials, fragrances, therapeutic agents, propellants, dyes, solvents, other emollients, conditioning agents, and vitamins. Optionally hydrophilic surfactants and other additives may be added to the antimicrobial composition. Typically, the formulations are essentially free of surfactants. In one embodiment, the compositions are essentially free of hydrophilic polymers, and water-soluble or water swellable polymers.

### Use of the Formulation

The antimicrobial compositions are useful for preoperative surgical, catheter, and i.v. antiseptic skin preparation, hand antiseptics and surgical scrubs. The antimicrobial compositions can be useful for preventing or reducing catheter related bloodstream infections. The compositions may be used to prevent surgical site infection by applying the compositions to the skin prior to surgery. These compositions can be applied to reduce the transient and normal flora of the skin. Repeated applications may be used to provide even higher efficacy on the skin.

The compositions can be applied using a variety of techniques including but not limited to: foamed applicators, cotton swabs, saturated swab sticks, saturated wipes, aerosols, sprays, brushes, and dips. The compositions may be contacted with the skin or inanimate object for 15 to 180 seconds and then allowed to dry. The compositions are useful for infection prevention products such as a preoperative antiseptic surgical preparations and antiseptic skin preparations used prior to catheterization. These compositions are useful when used in conjunction with medical adhesives, tapes, surgical drapes, and transparent dressing under wet or suboptimal conditions.

Since many of the compositions contain antimicrobials, it is important that they be dispensed in an efficacious and precise amount. The compositions can be dispensed in a discreet, substantially uniform amount using the dispensers disclosed in U.S. Pat. Nos. 5,897,031, and 5,799,841.

Although specific embodiments have been shown and described herein, it is understood that these embodiments are merely illustrative of the many possible specific arrangements that can be devised in application of the principles of the invention. Numerous and varied other arrangements can be devised in accordance with these principles by those of skill in the art without departing from the spirit and scope of the invention. The scope of the present invention should not be limited to the structures described in this application, but only by the structures described by the language of the claims and the equivalents of those structures.

### EXAMPLES

The chemicals used were sourced as follows:
20% CHG solution: Medichem S. A., Carrer de Fructuós Gelabert, 6, 08970 Sant Joan Despí, Barcelona, Spain

### Isopropanol: Sigma-Aldrich Company, 4353 E 49th St, Cleveland, Ohio 44125, USA

Acetyltributyl citrate: Sigma-Aldrich Company, 4353 E 49th St, Cleveland, Ohio 44125, USA
Ethocel 20 polymer: Dow Chemical Company, Midland, Mich. 48674, USA
N-(2-Hydroxyethyl)ethylenediaminetriacetic acid trisodium salt: Sigma-Aldrich Company, 4353 E 49th St, Cleveland, Ohio 44125, USA

### Starting Material:

Three hydroalcoholic chlorhexidine gluconate samples with varying amounts (in grams) of excipients were prepared for testing. The compositions of the three samples are shown below:

| Component (g) | A | B | C |
|---|---|---|---|
| 20% CHG solution | 6.1 | 6.1 | 6.1 |
| Water | 7.5 | 5.8 | 5.8 |
| Isopropanol | 36.4 | 32.1 | 32.1 |
| Acetyltributyl citrate | 0 | 6 | 3 |
| Ethocel 20 polymer | 0 | 0 | 3 |

All three samples were clear and free of particulates after preparation. The samples were stored at room temperature for 60 days. After about 30 days, tiny needle-like crystals of calcium gluconate were observed in all three solutions. The crystals initially increased in number but soon reached a steady state as observed visually, indicating the presence of a finite amount of calcium in the CHG solution.

### Example 1

The three samples from Example 1 were treated with varying levels of N-(2-Hydroxyethyl)ethylenediaminetriacetic acid trisodium salt (HEDTA.Na₃) (having a formation constant with calcium of greater than 10\hat{ }10) and stirred for three days. The solutions were then observed for crystal content. The results are shown below:

| | 12.5 ppm | 25 ppm | 50 ppm | 100 ppm |
|---|---|---|---|---|
| | (mg/kg) | (mg/kg) | (mg/kg) | (mg/kg) |
| Sample | HEDTA.Na₃ | HEDTA.Na₃ | HEDTA.Na₃ | HEDTA.Na₃ |
| A | Crystals | No crystals | No crystals | No crystals |
| B | Crystals | Crystals | Crystals | No crystals |
| C | Crystals | Crystals | Crystals | No crystals |

### Example 2

A chelator's formation constant with calcium impacts its ability to reduce the calcium crystal formation. Two well-known calcium chelating agents with relatively low formation constants with calcium: citric acid(CA) (having a formation constant with calcium of 10\hat{ }3.24) and sodium pyrophosphate(SP) (having a formation constant with calcium of 10\hat{ }4.95) were used in place of HEDTA.Na₃ in the example above and compared with HEDTA.Na₃.

| | 100 ppm | 200 ppm | 100 ppm | 200 ppm | 100 ppm |
|---|---|---|---|---|---|
| Sample | CA | CA | SP | SP | HEDTA.Na₃ |
| A | Crystals | Crystals | Crystals | Crystals | No crystals |
| B | Crystals | Crystals | Crystals | Crystals | No crystals |
| C | Crystals | Crystals | Crystals | Crystals | No crystals |

1-26. (canceled)

27. An antimicrobial composition comprising:
chlorhexidine;
a chlorhexidine-soluble solvent;
a chlorhexidine-insoluble solvent, wherein the chlorhexidine-insoluble solvent comprises at least 35 wt. % of the antimicrobial composition;
a chelating agent.

28. The antimicrobial composition of claim 27, wherein the chlorhexidine is selected from the group consisting of chlorhexidine digluconate, chlorhexidine diacetate, chlorhexidine dihydrochloride, chlorhexidine dimethosulfate, chlorhexidine dilactate, chlorhexidine diglucoheptonate, chlorhexidine diglycollate salts, and combinations thereof.

29. The antimicrobial composition of claim 27, wherein the chlorhexidine is present in an amount of least 0.05% by weight based on the total weight of the composition.

30. The antimicrobial composition of claim 27, wherein the chlorhexidine-soluble solvent comprises water and wherein the chlorhexidine-insoluble solvent comprises solvent is a C₂-C₅ lower alcohol.

31. The antimicrobial composition of claim 27, wherein the chlorhexidine-soluble solvent is at least 15 wt. % of the antimicrobial composition.

32. The antimicrobial composition of claim 27, wherein the chlorhexidine-soluble solvent is less than 25 wt. % of the antimicrobial composition.

33. The antimicrobial composition of claim 27, wherein the chlorhexidine-insoluble solvent comprises a hydrophobic polymer soluble or dispersible in the lower alcohol.

34. The antimicrobial composition of claim 33, wherein the hydrophobic polymer is selected from the group consisting of acrylates and its derivatives, cellulose and its derivatives, n-vinyl lactam copolymers and vinyl copolymers, and combinations of two or more of the foregoing.

35. The antimicrobial composition of claim 33, wherein the hydrophobic polymer is present in the antimicrobial composition in an amount of at least 2 wt. % based on the total weight of the antimicrobial composition.

36. The antimicrobial composition of claim 27, wherein the chlorhexidine-insoluble solvent is at least 60 wt. % of the antimicrobial composition.

37. The antimicrobial composition of claim 27, wherein the chlorhexidine-insoluble solvent is less than 80 wt. % of the antimicrobial composition.

38. The antimicrobial composition of claim 27, wherein the chlorhexidine-soluble solvent comprises less than 25 wt. % and the chlorhexidine-insoluble solvent comprises at least 75 wt. % of the antimicrobial composition.

39. The antimicrobial composition of claim 27, wherein the chelating agent is a polyanionic chelating agent.

40. The antimicrobial composition of claim 27, wherein the chelating agent is a soluble in at least one of the chlorhexidine-soluble solvent or the chlorhexidine-insoluble solvent.

41. The antimicrobial composition of claim 27, wherein the chelating agent has a formation constant with calcium of at least 10⁶ at neutral pH.

42. The antimicrobial composition of claim 27, wherein the chelating agent is present in the antimicrobial solution by less than 100 ppm.

43. The antimicrobial composition of claim 27, wherein the chelating agent is selected from the group consisting of glutamic acid N,N-diacetic acid, methylglycine N,N-diacetic acid, glucoheptonic acid, ethanoldiglycinic acid, diethylenetriaminepentaacetic acid, nitrilotriacetic acid, (N-(2-hydroxyethyl) ethylenediamine-N,N′,N′-triacetic acid trisodium salt, disodium ethylenediaminetetraacetic acid, or combinations thereof.

44. The antimicrobial composition of claim 27, further comprising a plasticizer.

45. The antimicrobial composition of claim 44, wherein the plasticizer is an emollient ester.

46. The antimicrobial composition of claim 27, wherein the antimicrobial composition is applied to a surface and is dried on the surface.
